(19) United States
(12) Patent Application Publication
(10) Pub. No.: US 2026/0009077 A1
(43) Pub. Date: Jan. 8, 2026

(54) COMPOSITIONS AND METHODS FOR TREATING HYPOTHYROIDISM

(71) Applicant: THE UNIVERSITY OF CHICAGO, Chicago, IL (US)

(72) Inventors: Antonio C. BIANCO, Chicago, IL (US); Matthew ETTLESON, Chicago, IL (US); Tatiana De Lourdes FONSECA, Chicago, IL (US)

(21) Appl. No.: 18/881,169

(22) PCT Filed: Jul. 5, 2023
(86) PCT No.: PCT/US2023/069628

§ 371 Date: Jan. 3, 2025

Related U.S. Application Data
(60) Provisional application No. 63/358,347, filed on Jul. 5, 2022.

(51) Int. Cl. C12Q 1/6883; A61K 31/192; A61K 31/197; A61K 35/55

(52) U.S. Cl. CPC C12Q 1/6883; A61K 31/192; A61K 31/197; A61K 35/55; C12Q 2600/106; C12Q 2600/156

(57) ABSTRACT

This disclosure relates to compositions and methods for treating hypothyroidism. Wherein a method of treating a patient for hypothyroidism, comprises: a) obtaining a sample from a patient containing DNA; b) determining a number of pre-defined single nucleotide polymorphisms (SNPs) contained in the DNA of the sample; c) administering to the patient a therapeutically effective amount of levothyroxine.

### CROSS-REFERENCE TO RELATED APPLICATIONS

This application claims the benefit of U.S. Provisional Application No. 63/358,347, filed Jul. 5, 2022, which is incorporated by reference herein in its entirety.

### BACKGROUND OF THE DISCLOSURE

### Field of Invention

This disclosure relates to compositions and methods for treating hypothyroidism.

### Technical Background

Hypothyroidism is a common disease that affects between 15-30 million individuals in the United States. The standard of care for treatment is the administration of daily tablets of levothyroxine, a synthetic form of thyroid hormone. This is a safe and effective treatment, which restores normal life in at least 80% of patients.

However, there remains a substantial number of patients that do not fully benefit from levothyroxine. These patients remain symptomatic, with detriments of quality of life, mood, and cognitive function. They also have difficulty in managing body weight. For these patients, it is recommended, on a trial basis, that a therapy that combines levothyroxine with liothyronine, a secondary more potent thyroid hormone, be administered. It is estimated that between 50-60% of such patients will benefit from the combination therapy. A recent statement from the American, European and British Thyroid Associations acknowledges and discusses this problem extensively (Jonklaas et al. Evidence-Based Use of Levothyroxine/Liothyronine Combinations in Treating Hypothyroidism: A Consensus Document. Thyroid. 2021 February; 31 (2): 156-182).

Currently, because the front line therapy for hypothyroidism is the administration of levothyroxine, it takes a considerable amount of time (˜6 months to 1 year) to determine whether or not this intervention will be successful because the symptoms of hypothyroidism are vague and hard to assess. As a result, patients suffer either because they do not receive combination therapy promptly or conversely because combination therapy could be prescribed for those patients who would fully benefit from the single therapy leading to potentially detrimental polypharmacy. Unfortunately, there are currently no tests available to predict how a patient will respond to levothyroxine. Therefore, there is a need to be able to differentiate between those patients who should receive the monotherapy (with levothyroxine) and those who should receive the combination therapy (with levothyroxine and liothyronine).

### SUMMARY OF THE DISCLOSURE

This disclosure describes compositions and methods for treating hypothyroidism.

In a first aspect, the present disclosure provides a method of treating a patient for hypothyroidism including a) obtaining a sample from a patient containing DNA; b) determining a number of pre-defined single nucleotide polymorphisms (SNPs) contained in the DNA of the sample; c) administering to the patient a therapeutically effective amount of levothyroxine if the DNA of the sample contains less than a threshold number of pre-defined SNPs; or d) administering to the patient a therapeutically effective amount of levothyroxine and a secondary therapeutic agent if the DNA of the sample contains a number of pre-defined SNPs that is equal to or greater than the threshold number of pre-defined SNPs.

In one embodiment of the first aspect, the sample is a blood sample, feces, urine, semen, saliva, hair, teeth, bone, or tissue (e.g., organ fragments or biopsies). In one embodiment of the first aspect, the pre-defined SNPs comprise one or more of rs2789337, rs2789334, rs1009080, exm-rs4949526, rs12059980, rs3908575, rs10157812, rs11166015, rs7519343, rs2295959, rs12468314, rs4853069, rs4508624, rs10490307, rs11896839, rs7576069, rs6757538, rs4954642, rs4664152, rs7565121, rs10204885, rs10497159, rs10191487, rs11894687, rs983733, rs6727865, exm-rs1569175, rs1569175, rs975894, rs10204710, rs3770536, rs4375854, rs6754280, rs4575672, rs962835, rs9826914, rs17023487, rs13094598, rs11720980, rs9845467, rs9859768, rs7626568, rs2197737, rs992130, rs6438269, rs7638568, rs10934299, rs9876228, rs1566695, rs7628226, rs6814444, rs3172524, rs2036204, rs13126556, rs4862815, rs12521751, rs3105807, rs2441115, rs32165, rs32153, rs6868612, rs2471106, rs1017998, rs10064975, rs1181968, rs1181962, rs13181777, rs4835970, rs2214077, rs7743224, rs1626976, rs873053, rs1948846, rs9363303, rs4537121, rs2350289, rs10498961, rs2250276, rs2787925, rs7742254, rs9489815, rs7801891, rs803157, rs1858826, rs6973845, rs855736, rs7790889, rs1521197, rs13259547, rs13270447, rs6989930, rs17152632, exm-rs10096633, rs7841189, rs17091905, rs2881123, rs2594797, rs4738532, rs654534, rs16929302, rs1563244, rs10099888, rs3133749, rs16886394, rs2597364, rs1863349, rs6984038, rs6577842, rs1411790, rs11794152, rs10812206, rs10758268, rs1418247, rs10904169, rs7919822, rs7095095, rs12244754, rs268300, rs4746509, rs2251717, rs1665672, rs4758435, rs11820608, rs11827258, rs879488, rs17134811, exm-rs10831284, rs10831284, rs519806, rs7102109, rs7943929, rs1001653, rs10745620, rs12311525, rs10778229, rs1961707, rs7966303, rs7966789, rs4759752, rs7308461, rs942462, rs7317245, rs9534199, rs2812219, rs1322960, rs2806947, rs7994590, rs17060536, rs1332384, rs616667, rs9570999, rs17792766, rs10149339, rs17112954, rs17096610, rs7141696, rs4313723, rs372543, rs17116830, rs17104036, rs10133270, rs11623037, rs482624, rs12909046, rs756426, rs150063, rs235807, rs1837024, rs372627, rs6503121, rs4925112, rs4789011, rs7222391, rs12326169, rs1623173, rs4510110, rs2069124, rs8096215, rs4432372, rs2591591, rs2591593, rs2547072, rs953551, rs2591594, rs2547074, rs2547076, rs2591595, rs1862462, rs2591597, rs7245949, rs7245960, rs1609460, rs1609459, rs10402812, rs2384687, rs8117167, rs219865, rs6039529, rs6081694, rs2822554, rs8134718, rs914136, rs2832341, rs2244305, rs765429, rs4413240, rs9620647, rs5752454, and rs17659494.

In any of the preceding aspects or embodiments, the threshold number of pre-defined SNPs is about 15. In any of the preceding aspects or embodiments, the threshold number of pre-defined SNPs is about 107. In any of the preceding aspects or embodiments, the secondary therapeutic agent comprises one or more of liothyronine, desiccated thyroid extract, T3 sulphate or poly-zinc-liothyronine, and T3 analogues. In one embodiment, the T3 analogues comprise eprotirome and sobetirome. In one embodiment, the desiccated thyroid extract contains levothyroxine and liothyronine derived from an animal source.

In any of the preceding aspects or embodiments, the method further includes (e) improving one or more of quality of life, mood, and cognitive parameters in the patient.

In a second aspect, the present disclosure provides a method of improving treatment to a patient for hypothyroidism including a) obtaining a sample from a patient containing DNA; b) determining a number of pre-defined single nucleotide polymorphisms (SNPs) contained in the DNA of the sample; c) administering to the patient a therapeutically effective amount of levothyroxine if the DNA of the sample contains less than a threshold number of pre-defined SNPs; or d) administering to the patient a therapeutically effective amount of levothyroxine and a secondary therapeutic agent if the DNA of the sample contains a number of pre-defined SNPs that is equal to or greater than the threshold number of pre-defined SNPs; and (e) one or more of i) reducing incidents of the patient receiving an ineffective treatment for hypothyroidism, ii) reducing the time required for the patient to receive an effective treatment for hypothyroidism, and iii) reducing healthcare costs associated with receiving treatment for hypothyroidism.

In one embodiment of the second aspect, the sample is a blood sample, feces, urine, semen, saliva, hair, teeth, bone, or tissue. In one embodiment of the second aspect or any embodiment thereof, the pre-defined SNPs comprise one or more of rs2789337, rs2789334, rs1009080, exm-rs4949526, rs12059980, rs3908575, rs10157812, rs11166015, rs7519343, rs2295959, rs12468314, rs4853069, rs4508624, rs10490307, rs11896839, rs7576069, rs6757538, rs4954642, rs4664152, rs7565121, rs10204885, rs10497159, rs10191487, rs11894687, rs983733, rs6727865, exm-rs1569175, rs1569175, rs975894, rs10204710, rs3770536, rs4375854, rs6754280, rs4575672, rs962835, rs9826914, rs17023487, rs13094598, rs11720980, rs9845467, rs9859768, rs7626568, rs2197737, rs992130, rs6438269, rs7638568, rs10934299, rs9876228, rs1566695, rs7628226, rs6814444, rs3172524, rs2036204, rs13126556, rs4862815, rs12521751, rs3105807, rs2441115, rs32165, rs32153, rs6868612, rs2471106, rs1017998, rs10064975, rs1181968, rs1181962, rs13181777, rs4835970, rs2214077, rs7743224, rs1626976, rs873053, rs1948846, rs9363303, rs4537121, rs2350289, rs10498961, rs2250276, rs2787925, rs7742254, rs9489815, rs7801891, rs803157, rs1858826, rs6973845, rs855736, rs7790889, rs1521197, rs13259547, rs13270447, rs6989930, rs17152632, exm-rs10096633, rs7841189, rs17091905, rs2881123, rs2594797, rs4738532, rs654534, rs16929302, rs1563244, rs10099888, rs3133749, rs16886394, rs2597364, rs1863349, rs6984038, rs6577842, rs1411790, rs11794152, rs10812206, rs10758268, rs1418247, rs10904169, rs7919822, rs7095095, rs12244754, rs268300, rs4746509, rs2251717, rs1665672, rs4758435, rs11820608, rs11827258, rs879488, rs17134811, exm-rs10831284, rs10831284, rs519806, rs7102109, rs7943929, rs1001653, rs10745620, rs12311525, rs10778229, rs1961707, rs7966303, rs7966789, rs4759752, rs7308461, rs942462, rs7317245, rs9534199, rs2812219, rs1322960, rs2806947, rs7994590, rs17060536, rs1332384, rs616667, rs9570999, rs17792766, rs10149339, rs17112954, rs17096610, rs7141696, rs4313723, rs372543, rs17116830, rs17104036, rs10133270, rs11623037, rs482624, rs12909046, rs756426, rs150063, rs235807, rs1837024, rs372627, rs6503121, rs4925112, rs4789011, rs7222391, rs12326169, rs1623173, rs4510110, rs2069124, rs8096215, rs4432372, rs2591591, rs2591593, rs2547072, rs953551, rs2591594, rs2547074, rs2547076, rs2591595, rs1862462, rs2591597, rs7245949, rs7245960, rs1609460, rs1609459, rs10402812, rs2384687, rs8117167, rs219865, rs6039529, rs6081694, rs2822554, rs8134718, rs914136, rs2832341, rs2244305, rs765429, rs4413240, rs9620647, rs5752454, and rs17659494. In one embodiment of the second aspect or any embodiment thereof, the threshold number of pre-defined SNPs is about 15. In one embodiment of the second aspect or any embodiment thereof, the threshold number of pre-defined SNPs is about 107. In one embodiment of the second aspect or any embodiment thereof, the secondary therapeutic agent comprises one or more of liothyronine, desiccated thyroid extract, T3 sulphate or poly-zinc-liothyronine, and T3 analogues. In one embodiment of the second aspect or any embodiment thereof, the T3 analogues comprise eprotirome and sobetirome. In one embodiment of the second aspect, the desiccated thyroid extract contains levothyroxine and liothyronine derived from an animal source.

In a third aspect, the present disclosure provides a method of treating a patient for hypothyroidism including a) obtaining a sample from a patient containing DNA; b) determining a number of pre-defined single nucleotide polymorphisms (SNPs) contained in the DNA of the sample; c) administering to the patient a therapeutically effective amount of levothyroxine if the DNA sample contains less than a threshold number of pre-defined SNPs; or d) administering to the patient a therapeutically effective amount of levothyroxine and a secondary therapeutic agent if the DNA of the sample contains a number of SNPs that is equal to or greater than the threshold number of pre-defined SNPs, wherein when the number of pre-defined SNPs is greater than the threshold number of pre-defined SNPs, the number of pre-defined SNPs present in the DNA of the sample correlates to a relative dose of the secondary therapeutic agent required to effectively treat hypothyroidism in the patient. In one embodiment, the method identifies those patients that will require relatively higher doses of the second therapeutic agent used in combination with levothyroxine. In some embodiments, the number of pre-defined SNPs present in the DNA of the sample is instructive of a relative dose of the secondary therapeutic agent required to effectively treat hypothyroidism in the patient.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. Manhattan plot showing the strength of the association between the single nucleotide polymorphisms (SNPs) located in each chromosome and the trait. The line represents a p-value threshold of 1.0×10⁻⁵. The SNP with the lowest p-value in each chromosome is labeled.

FIG. 2. Quantile-Quantile (QQ) plot of distribution of observed p-values of the association study versus and distribution of the expected p-values.

FIG. 3. ROC curve of the base SNP profile score (with top 209 SNPs) within the study population. An optimal threshold score of 107 was identified.

FIG. 4. ROC curve of the base SNP profile score (with top 15 SNPs) within the study population. An optimal threshold score of 12 was identified.

### DETAILED DESCRIPTION

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to that amount of a therapeutic agent, such as levothyroxine, liothyronine, and/or desiccated thyroid extract, which when administered to a subject, is sufficient to effect treatment (e.g., improve or eradicate symptoms) for a disease or disorder described herein, such as, for example hypothyroidism. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of the disease or disorder or one or more symptoms of the disease or disorder.

“Subject” refers to a warm blooded animal such as a mammal, preferably a human, which is afflicted with, or has the potential to be afflicted with one or more diseases and disorders described herein. The term subject can also refer to a patient, such as an individual that has been diagnosed as having hypothyroidism.

“Pharmaceutical composition” as used herein refers to a composition that includes one or more therapeutic agents disclosed herein, such as levothyroxine, liothyronine, and/or desiccated thyroid extract, a pharmaceutically acceptable carrier, a solvent, an adjuvant, and/or a diluent, or any combination thereof.

As used herein, the term “hypothyroidism” refers to a condition or disease state in which an individual has underactive or non-active thyroid function or an individual that was born without a thyroid gland or in whom the thyroid gland was surgically removed or destroyed with radioactive iodine. This condition is characterized by cognitive and metabolic impairments.

In view of the present disclosure, the methods and compositions described herein can be configured by the person of ordinary skill in the art to meet the desired need. In general, the disclosed materials and methods provide improvements in treating hypothyroidism as described herein.

### Overview

The present disclosure is directed to compositions and methods for treating hypothyroidism. While effective treatments for patients suffering from hypothyroidism are available, because of the multifaceted symptomatic presentation of the disease coupled with the relatively slow onset of efficacy of the front line therapy (i.e., oral levothyroxine), many patients are not receiving ideal or even proper care. A root cause of this problem is the previous inability of clinicians to determine which patients would be adequately treated by the front line therapy versus those who would require an augmented therapy regimen that combines a secondary therapeutic agent (e.g., liothyronine) with levothyroxine or desiccated thyroid extract, which contains both levothyroxine and liothyronine. Therefore, the technology presented herein allows for the identification of patients with this trait, i.e., those that fail therapy with levothyroxine and would benefit from combination therapy.

This technology is highly significant because it can improve patient care for thousands to millions of individuals in the U.S. and throughout the world. Because the symptoms are vague and hard to assess, without an objective diagnostic tool patient care is compromised, with combination therapy being under-utilized for patients that really need it (remain symptomatic) and over-utilized for patients that do not need it (symptoms unrelated to hypothyroidism).

### Methods

In one embodiment, the present disclosure provides a method for customization of the treatment of hypothyroidism. The method can include the steps of obtaining a DNA-containing sample from a patient, studying in that sample the presence or absence of a series of one or more pre-defined single nucleotide polymorphisms (SNPs) contained in a SNP master list, and administering to the patient a customized treatment depending on the number of SNPs from the master list that were present in the patient's sample. If the patient sample contains a number of pre-defined SNPs (from the master list) that is equal to or greater than a pre-defined critical number of SNPs, then the patient will receive a therapeutically effective amount of levothyroxine and liothyronine, or desiccated thyroid extract. Alternatively, if the patient sample contains a number of pre-defined SNPs (from the master list) that is less than the pre-defined critical number of SNPs, then the patient will receive a therapeutically effective amount of levothyroxine alone.

In one embodiment, the patient can be newly diagnosed with hypothyroidism or can be a patient who has been previously treated for hypothyroidism.

In one embodiment, the sample to taken from the patient is a DNA sample that can be obtained with a swab from several common sources including blood, feces, urine, semen, saliva, hair, teeth, bone, and tissue (organ fragments).

In one embodiment, SNPs are the most frequent sequence variations in the human DNA sequence, occurring approximately once every 100 to 300 base pairs. Apart from identical twins, each individual has a unique combination of nucleotides at these positions. Thus, a SNP profile provides a kind of unique fingerprint.

In some embodiments, a threshold number of pre-defined SNPs for the present disclosure is about 15, but other values are contemplated herein (e.g., any number from 2 to 209). In some embodiments, the threshold number of pre-defined SNPs is about or equal to 100, 101, 102, 103, 104, 105, 106, 107, 108, 109, or 110.

In one embodiment, there are two forms of combination therapy: (i) co-administration of synthetic levothyroxine and the secondary therapeutic agent liothyronine, at the same time or different times during the day, (ii) or desiccated thyroid extract, which contains both levothyroxine and liothyronine from an animal source. Additional examples of secondary agents for treating hypothyroidism include any other molecules that after metabolization, in the intestine or liver or in any other part of the body will generate liothyronine, such as T3 sulphate or poly-zinc-liothyronine. T3 analogues such as eprotirome and sobetirome are also examples of secondary therapeutic agents.

In another embodiment, the present disclosure provides a method of treating a patient for hypothyroidism. The method includes the steps of obtaining a DNA sample from a patient, determining a SNP profile of the patient's sample, withholding administration to the patient of a therapeutically effective amount of levothyroxine and a secondary therapeutic agent if the patient's DNA sample contains less than a critical (e.g., threshold) number of SNPs present in the SNP master list, and administering to the patient a therapeutically effective amount of levothyroxine only.

In another embodiment, the present disclosure provides a method treating a patient for hypothyroidism. The method includes the steps of obtaining a DNA sample from a patient, determining a SNP profile of the patient's sample, and administering to the patient a therapeutically effective amount of levothyroxine and a secondary therapeutic agent if the patient's DNA sample contains a number of SNPs that is equal to or greater than a critical number of SNPs present in the SNP master list.

Additional methods are contemplated herein.

### Compositions

In some embodiments, pharmaceutical compositions contemplated herein include a therapeutically effective amount of levothyroxine, liothyronine, and/or desiccated thyroid extract. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, levothyroxine, liothyronine, and/or desiccated thyroid extract is administered orally, and the subject is dosed on a daily basis.

The therapeutic agents (also referred to as “compounds” herein) described herein (e.g., levothyroxine, liothyronine, and/or desiccated thyroid extract), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of levothyroxine, liothyronine, and/or desiccated thyroid extract or other contemplated therapeutic agent can be in the range of from about 0.0001 mg/kg/day, about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Determination of Treatment Outcomes in Patients with Hypothyroidism

### Introduction

Hypothyroidism is a prevalent disease, affecting approximately between 5 and 10% of the U.S. population. Primary hypothyroidism is caused by the destruction of the thyroid gland, commonly associated with Hashimoto's thyroiditis, an autoimmune disease. It can also be the result of surgical removal of the thyroid gland or treatment with radioactive iodine. Patients with hypothyroidism have low energy and may gain weight, experience fatigue, retain fluid, and might also have cognitive problems such as poor memory and difficulty concentrating during routine, daily activities. Overall, these symptoms are the result of low blood levels of thyroid hormones, due to the malfunctioning of the thyroid gland. Contrary to some other diseases, treatment of hypothyroidism is not aimed at restoring thyroid gland function. Instead, it aims to normalize thyroid hormone levels in the blood by administering replacement thyroid hormones to patients (1).

Treatment of hypothyroidism was conceived around 1890 and consisted of the daily administration of a tablet containing a pig's thyroid extract, which contained the two main hormones produced by the thyroid gland: T4 and T3 (2). In 1970, scientists in the U.S. discovered that, after absorption into the circulation, T4 (the predominant form of thyroid hormone released by the thyroid gland) is converted to T3 (the active form of thyroid hormone), which was interpreted as “no need to treat patients with a treatment containing T3” (3). Since then, the standard of care has been treatment with T4 only (4). However, the switch in treatment resulted in unintended consequences. A small proportion of patients, between 15-20%, quickly noticed the reemergence of hypothyroid symptoms upon being switched, despite receiving the appropriate doses of T4. They demanded to be placed back on the old treatment containing both T4 and T3 (5).

This crisis evolved over the next several decades, with most doctors and medical associations advocating for the standard of care with T4 only. The controversy was resolved provisionally between 2012 and 2014, when clinical guidelines in Europe and the U.S. changed, advocating for a trial of the “old treatment” for those patients that did not feel well on T4 alone (6). Patients with hypothyroidism that remain symptomatic despite treatment with T4 represent a growing problem for both primary care and specialist physicians, including endocrinologists. Because most patients do well on T4 alone, the practice is to indeed use T4 to treat any patient with hypothyroidism. If, after several months of adjusting the T4 dose and repeated laboratory tests the patient still does not feel well, then therapy with T4+T3 may be attempted. However, switching patients from T4 alone to T4+T3 is time-consuming (up to a year), results in additional healthcare costs (e.g. additional thyroid function testing), and there is no guarantee that the patient will feel better. Estimates place the success rate of therapy with T4+T3 for symptomatic patients with hypothyroidism at approximately 60%. Current guidelines recommend that if the patient does not symptomatically improve after a trial of T4+T3 therapy, then the patient should be restarted on T4 therapy alone and other causes of symptoms should be investigated.

Patients and physicians alike are frustrated with the current situation. Therefore, there is an urgent need for the development of an objective diagnostic method that can identify which patients with residual symptoms of hypothyroidism while on T4 would benefit from switching to therapy containing both T4 and T3. Moreover, the present disclosure will enable clinicians 1) to appropriately treat newly diagnosed patients with more appropriate therapeutic regimens (in terms of specific therapeutic agents a patient should receive) and 2) to potentially determine therapeutic effective dosing for therapeutic agents to be administered.

### Materials and Methods

The patients' DNA samples used in the present studies were obtained from the Walter Reed Medical Military Center. There, investigators conducted a double-blind, randomized, prospective clinical trial with 90 patients having hypothyroidism. A total of 75 participants completed a crossover trial during which each participant completed 3 study arms in a randomized order: 16 weeks taking levothyroxine, 16 weeks taking combination therapy (levothyroxine+liothyronine), and 16 weeks taking desiccated thyroid extract (7). At the end of each study arm, thyroid function tests were obtained from each participant, and data were collected on thyroid disease symptoms via the thyroid symptom questionnaire (TSQ). The TSQ consists of 12 questions, each scored from 0 (not experiencing that symptom at all) to 3 (experiencing that symptom much more than usual), for a total of 36 points (higher numbers=more symptoms). Each participant produced 4 total scores (baseline and after each arm) throughout the study period. Participants were also assessed for quality of life, mood, and cognition to identify those patients that remained symptomatic (7).

Of the 75 participants in the original clinical trial, blood samples from 68 participants were obtained for gene sequencing. All samples underwent whole-exome sequencing (Infinium OmniExpress microarray) performed by the Broad Institute, Cambridge MA. Data for approximately 800,000 SNPs from each patient were processed using PLINK v1.9. During standard quality control measures, 4 additional samples were excluded for a final study sample of 64 participants (12 males, 52 females).

A genome-wide association study was performed to determine the association between individual SNPs and symptomatic response to combination therapy. Cases were defined as participants who had a TSQ score following the levothyroxine trial arm that was greater than the median score (15) and had a lower score (i.e. fewer symptoms) following the combination therapy arm compared to the levothyroxine arm. In other words, any participants that had above-average symptoms on levothyroxine which improved on combination therapy were defined as a case. All other participants were defined as controls. In total, there were 19 cases and 45 controls. Manhattan and QQ plots were generated via PLINK and R Studio.

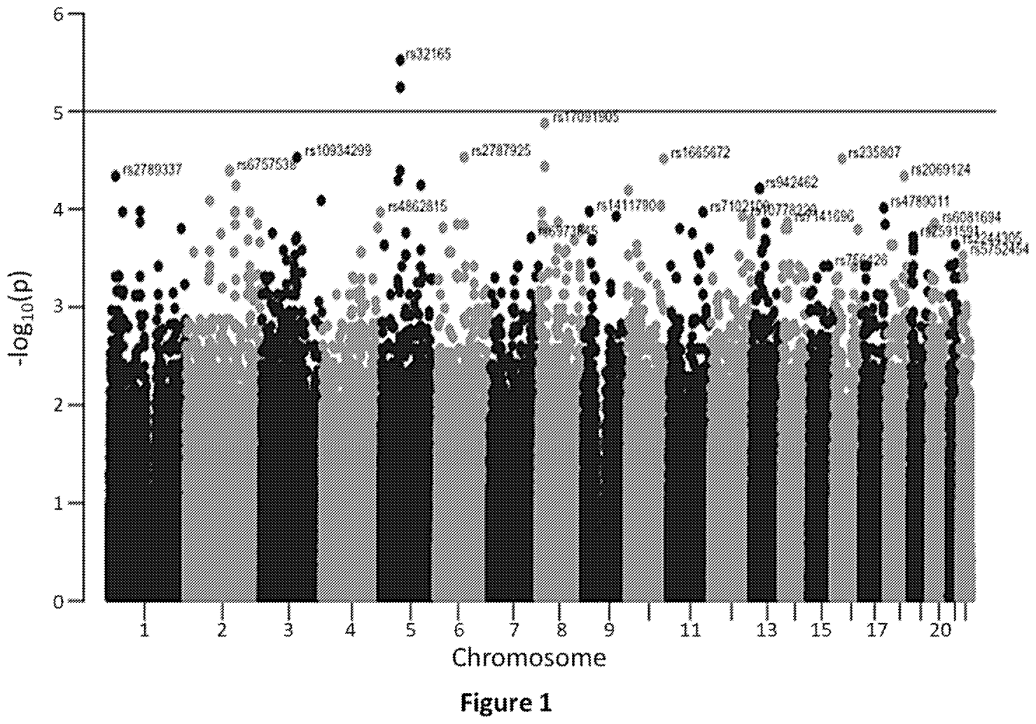

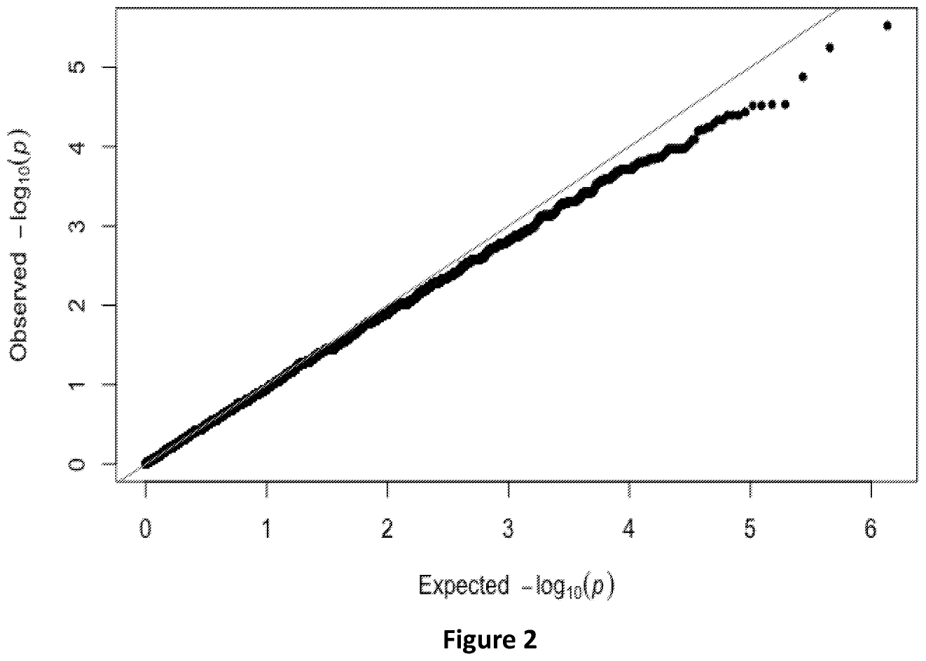

The strength of association between each SNP and the trait of interest was calculated and plotted in a Manhattan plot (FIG. 1). A QQ plot was generated to determine if the observed quantile distribution of SNP-trait association p-values varied from the expected distribution of p-values (FIG. 2). Next, all SNPs were ordered by strength of association (adjusted p-value). A p-value threshold of 5.0×10⁻⁴ was selected, identifying the top 209 SNPs with the strongest associations with the trait of interest. These 209 SNPs formed the master list of SNPs (Table 1, see below).

Because each one of these SNPs exhibited a statistically significant association with the trait (p-value<5.0×10⁻⁴), each one of these SNPs can be used to predict the presence of the trait in a given patient. However, by combining the predictive value of two or more SNPs, we can increase both the sensitivity and specificity of the prediction. Thus, we compared the specificity and sensitivity of the whole master list (containing 209 SNPs) with multiple subsets of SNPs contained in the master list.

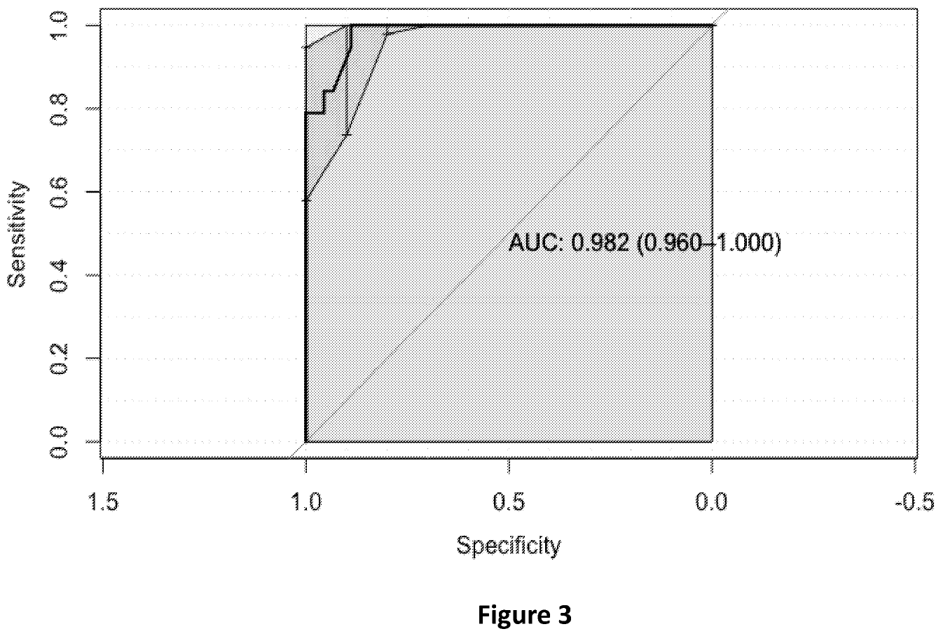

While working with the complete set of SNPs (master list), each participant was assigned a score based on the presence or absence of each of the 209 SNPs, with a binary ‘yes/no’ score for each SNP (score=0 for 0 copies of the SNP, score=1 for 1 or 2 copies of the SNP). A receiver operating characteristic (ROC) curve (FIG. 3) was generated to determine the score threshold which optimized sensitivity and specificity of the base SNP profile score.

Next, we created subsets of SNPs and tested their ability to predict an association with the trait of interest. The SNP-set Sequence Kernel Association Test (SKAT) was utilized to determine the strength of association between each set and the trait. SNP sets were tested sequentially (the first set consisted of the two most highly associated SNPs with the trait, the next set consisted of the top 3 SNPs, and so on until the SNP set with the lowest p-value was found. Similar to what was done with the complete master list, the presence or absence of each copy of the SNPs was scored (score=0 for 0 copies of the SNP, score=1 for 1 copy, score=2 for 2 copies, for a max score of 30), resulting in a predictor score for each participant.

An ROC curve was generated using each participant's predictor score (calculated based on the master list of SNPs and for the subsets of SNPs) to determine the score threshold with optimal sensitivity and specificity. Given the case prevalence within a specific patient population (i.e., the percentage of patients with above-average symptom scores on levothyroxine that improve with combination therapy), positive and negative predictive values for any given score threshold could be determined.

### Results

A case-control genome wide association study of the included 64 participants was performed, with the individual SNP p-values with symptomatic improvement with combination therapy plotted in FIG. 1. The QQ plot (FIG. 2) demonstrated that the observed quantile distribution of p-values was similar to the expected quantile distribution. A total of 209 individual SNPs met the p-value threshold of 5.0×10⁻⁴. A base SNP profile score was calculated for each participant based on the presence of each SNP. An ROC curve (FIG. 3) was generated, which identified an optimal threshold score of 107, with a sensitivity and specificity of 88.9% and 94.7% (AUC 0.982, 95% CI [0.960-0.999), respectively, in the study population. Extrapolating to a patient population with a trait prevalence of 20%, the positive predictive value (PPV) of the test is estimated to be 81% and the negative predictive value (NPV) is estimated to be 97%.

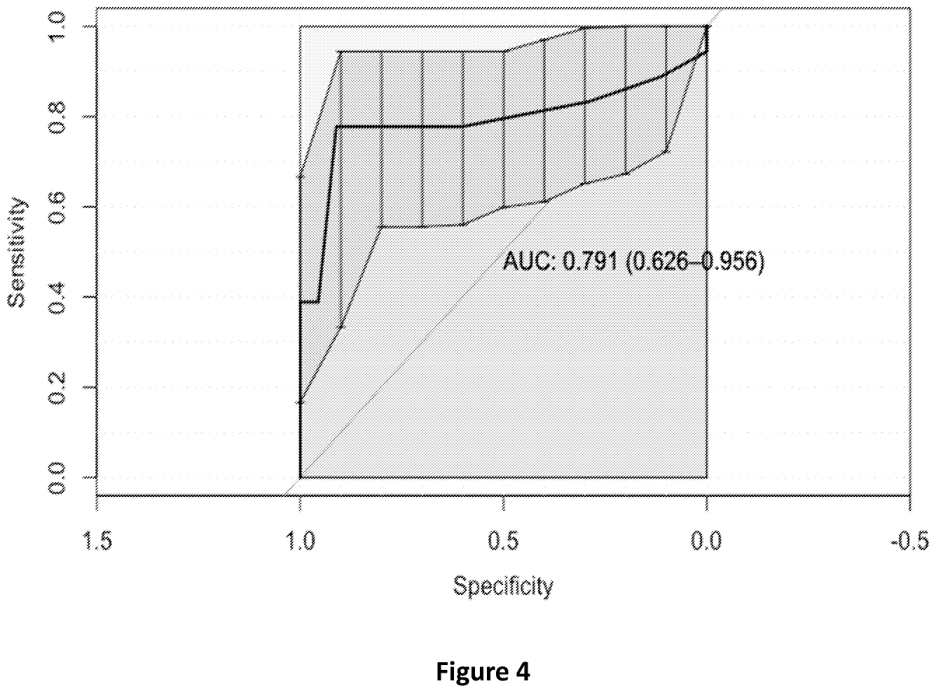

Using the SKAT method to determine the association between SNP subsets and the trait, we identified a subset of 15 SNPs from the top 209 SNPs that represented the SNP set with the strongest association with the trait of interest (p-value=1.09×10⁻⁹). An ROC curve (FIG. 4) was generated, which identified a SNP subset profile score threshold of 12, with a sensitivity of 77.8% and specificity of 91.1% (AUC 0.791, 95% CI [0.626-0.956) in the study population. Extrapolating to a patient population with a trait prevalence of 20%, the positive predictive value (PPV) of the test is estimated to be 69% and the negative predictive value (NPV) is estimated to be 94%.

The predictive value of the complete list of 209 SNPs was marginally superior to that of the subset of 15 SNPs. It is expected that if other subsets of SNPs from the master list were to be used, their predictive value would also gravitate around the figures we obtained.

As an illustrative example, a patient presents to her physician because she has increased fatigue and has gained 10 lbs over the last few months. She was diagnosed with Hashimoto's thyroiditis 6 months ago and has been taking an appropriate dose of levothyroxine since the diagnosis. The physician confirms that her thyroid function tests (thyrotropin and free thyroxine) are within the normal range with a simple blood test. The physician then sends that sample for genetic analysis of the 209 identified predictor SNPs. The predictor score returns at 121 (out of 209), meaning the patient has 120 polymorphisms within the 209 SNP locations found to be associated with better response to T4+T3 therapy. The critical value for the list of 209 SNPs is 107. Thus, the physician follows up with the patient and explains that about “8 out of 10 patients with hypothyroidism and your genetic profile will have a better response to T3-containing therapy.” As a result, the patient and physician together decide to trial the patient on combination therapy. The patient returns after several weeks (e.g., 8 weeks) and her energy levels are improved and her weight has stabilized while her thyroid function tests remain within normal limits.

Alternatively, the physician could also have sent that sample for genetic analysis of the highly associated set of 15 SNPs and obtained similar results. Given that all of the 209 SNPs were (statistically) significantly associated with the trait, any combination of any group of SNPs within these 209 SNPs would provide excellent prediction value.

### CONCLUSION

While the treatment of hypothyroidism with levothyroxine as thyroid hormone replacement is an effective therapy for many patients, there remains a large group of patients that are dissatisfied with treatment due to persistent symptoms. These patients represent a challenge for clinicians because the symptoms are non-specific and experience with other forms of thyroid hormone is limited. Therefore, more effective treatment with T3 is offered to such patients after significant delay or not at all. Alternatively, there may be patients with persistent symptoms unrelated to the thyroid that are offered T3-containing therapy that ultimately will provide no additional benefit. The role of the inventive predictor assay disclosed herein is to provide physicians with an increased degree of certainty that such patients will or will not respond to T3-containing therapy. In turn, this will help physicians provide more targeted therapy for hypothyroidism and, most importantly, provide patients with a better thyroid hormone replacement profile for their individual physiologic needs.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

### REFERENCES

1. Chaker L, Bianco A C, Jonklaas J, and Peeters R P. Hypothyroidism. Lancet. 2017.
2. McAninch E A, and Bianco A C. The History and Future of Treatment of Hypothyroidism. Annals of internal medicine. 2016; 164 (1): 50-6.
3. Braverman L E, Ingbar S H, and Sterling K. Conversion of thyroxine (T4) to triiodothyronine (T3) in athyreotic subjects. Journal of Clinical Investigation. 1970; 49:855-64.
4. Jonklaas J, Bianco A C, Bauer A J, Burman K D, Cappola A R, Celi F S, et al. Guidelines for the treatment of hypothyroidism: prepared by the american thyroid association task force on thyroid hormone replacement. Thyroid: official journal of the American Thyroid Association. 2014; 24 (12): 1670-751.
5. Hegedus L, Bianco A C, Jonklaas J, Pearce S H, Weetman A P, and Perros P. Primary hypothyroidism and quality of life. Nature reviews Endocrinology. 2022; 18 (4): 230-42.
6. Jonklaas J, Bianco A C, Cappola A R, Celi F S, Fliers E, Heuer H, et al. Evidence-Based Use of Levothyroxine/Liothyronine Combinations in Treating Hypothyroidism: A Consensus Document. Thyroid: official journal of the American Thyroid Association. 2021; 31 (2): 156-82.
7. Shakir M K M, Brooks D I, McAninch E A, Fonseca T L, Mai V Q, Bianco A C, et al. Comparative effectiveness of levothyroxine, desiccated thyroid extract, and levothyroxine+Liothyronine in hypothyroidism. The Journal of clinical endocrinology and metabolism. 2021.

TABLE 1

| | SNP | | | | | |
|---|---|---|---|---|---|---|
| SNP NO. | COUNT | CHROMOSOME | POSITION | SNP ID | REF | ALT |
| 1 | 8439 | 1 | 19100178 | rs2789337 | G | A |
| 2 | 8440 | 1 | 19100436 | rs2789334 | A | G |
| 3 | 13345 | 1 | 30431560 | rs1009080 | G | A |
| 4 | 13346 | 1 | 30432219 | exm-rs4949526 | T | C |
| 5 | 18080 | 1 | 42356812 | rs12059980 | A | C |
| 6 | 36865 | 1 | 98863760 | rs3908575 | C | T |
| 7 | 36870 | 1 | 98883541 | rs10157812 | A | G |
| 8 | 36874 | 1 | 98904871 | rs11166015 | A | C |
| 9 | 49958 | 1 | 158403819 | rs7519343 | C | T |
| 10 | 76198 | 1 | 231954682 | rs2295959 | C | T |
| 11 | 90740 | 2 | 23347247 | rs12468314 | G | A |
| 12 | 109347 | 2 | 75116627 | rs4853069 | C | T |
| 13 | 109463 | 2 | 75479258 | rs4508624 | A | G |
| 14 | 109465 | 2 | 75482223 | rs10490307 | C | T |
| 15 | 109721 | 2 | 76088361 | rs11896839 | C | T |
| 16 | 117560 | 2 | 111830273 | rs7576069 | T | G |
| 17 | 124987 | 2 | 139677323 | rs6757538 | A | C |
| 18 | 124994 | 2 | 139709252 | rs4954642 | C | T |
| 19 | 128684 | 2 | 154549105 | rs4664152 | C | T |
| 20 | 129333 | 2 | 157731753 | rs7565121 | C | T |
| 21 | 129338 | 2 | 157758173 | rs10204885 | C | T |
| 22 | 129341 | 2 | 157785456 | rs10497159 | A | G |
| 23 | 129344 | 2 | 157797836 | rs10191487 | A | G |
| 24 | 129930 | 2 | 159792033 | rs11894687 | C | T |
| 25 | 141582 | 2 | 199479235 | rs983733 | G | A |
| 26 | 141670 | 2 | 200029496 | rs6727865 | G | A |
| 27 | 141841 | 2 | 201021954 | exm-rs1569175 | T | C |
| 28 | 141842 | 2 | 201021954 | rs1569175 | T | C |
| 29 | 143383 | 2 | 205645815 | rs975894 | C | T |
| 30 | 143385 | 2 | 205646988 | rs10204710 | T | C |
| 31 | 146666 | 2 | 216838820 | rs3770536 | A | G |
| 32 | 151731 | 2 | 230919116 | rs4375854 | C | T |
| 33 | 151732 | 2 | 230919938 | rs6754280 | G | A |
| 34 | 154637 | 2 | 238070572 | rs4575672 | T | C |
| 35 | 161787 | 3 | 10764022 | rs962835 | G | T |
| 36 | 161788 | 3 | 10771533 | rs9826914 | G | A |
| 37 | 168055 | 3 | 29482712 | rs17023487 | G | A |
| 38 | 169869 | 3 | 35734231 | rs13094598 | C | T |
| 39 | 183777 | 3 | 71505650 | rs11720980 | C | A |
| 40 | 186127 | 3 | 81614473 | rs9845467 | G | A |
| 41 | 190361 | 3 | 106231571 | rs9859768 | T | C |
| 42 | 190388 | 3 | 106335082 | rs7626568 | C | T |
| 43 | 191141 | 3 | 108677821 | rs2197737 | C | A |
| 44 | 191538 | 3 | 110676677 | rs992130 | A | G |
| 45 | 193222 | 3 | 115289876 | rs6438269 | T | C |
| 46 | 193223 | 3 | 115290089 | rs7638568 | G | A |
| 47 | 193229 | 3 | 115301990 | rs10934299 | C | T |
| 48 | 198984 | 3 | 131399961 | rs9876228 | G | A |
| 49 | 198990 | 3 | 131414382 | rs1566695 | C | A |
| 50 | 216366 | 3 | 193748075 | rs7628226 | C | T |
| 51 | 244127 | 4 | 98441586 | rs6814444 | C | T |
| 52 | 250789 | 4 | 125587561 | rs3172524 | G | T |
| 53 | 250809 | 4 | 125620031 | rs2036204 | A | G |
| 54 | 263591 | 4 | 180036877 | rs13126556 | T | C |
| 55 | 267012 | 4 | 188479049 | rs4862815 | T | G |
| 56 | 271811 | 5 | 9896255 | rs12521751 | G | A |
| 57 | 282487 | 5 | 54392373 | rs3105807 | C | T |
| 58 | 284468 | 5 | 61747500 | rs2441115 | G | A |
| 59 | 284486 | 5 | 61854650 | rs32165 | C | T |
| 60 | 284491 | 5 | 61867920 | rs32153 | T | C |
| 61 | 285806 | 5 | 67185222 | rs6868612 | G | A |
| 62 | 289387 | 5 | 79254118 | rs2471106 | A | G |
| 63 | 289392 | 5 | 79263211 | rs1017998 | A | G |
| 64 | 289394 | 5 | 79277215 | rs10064975 | A | G |
| 65 | 301667 | 5 | 128354053 | rs1181968 | C | T |
| 66 | 301678 | 5 | 128386801 | rs1181962 | A | G |
| 67 | 301684 | 5 | 128408502 | rs13181777 | A | G |
| 68 | 301708 | 5 | 128463532 | rs4835970 | G | A |
| 69 | 303947 | 5 | 136028077 | rs2214077 | A | C |
| 70 | 327544 | 6 | 19643688 | rs7743224 | A | G |
| 71 | 343561 | 6 | 38698580 | rs1626976 | T | C |
| 72 | 346862 | 6 | 45847115 | rs873053 | C | T |
| 73 | 351023 | 6 | 63883378 | rs1948846 | A | G |
| 74 | 351360 | 6 | 65687330 | rs9363303 | C | T |
| 75 | 351374 | 6 | 65751371 | rs4537121 | C | T |
| 76 | 351375 | 6 | 65756221 | rs2350289 | T | G |
| 77 | 357057 | 6 | 88369843 | rs10498961 | G | A |
| 78 | 357076 | 6 | 88397755 | rs2250276 | T | C |
| 79 | 357078 | 6 | 88410955 | rs2787925 | A | G |
| 80 | 358484 | 6 | 92604703 | rs7742254 | G | A |
| 81 | 365516 | 6 | 120133826 | rs9489815 | G | A |
| 82 | 389228 | 7 | 17133514 | rs7801891 | A | C |
| 83 | 409576 | 7 | 90739777 | rs803157 | C | T |
| 84 | 410411 | 7 | 93349015 | rs1858826 | C | T |
| 85 | 422427 | 7 | 134436060 | rs6973845 | A | G |
| 86 | 427270 | 7 | 149363363 | rs855736 | C | A |
| 87 | 430947 | 7 | 158102524 | rs7790889 | C | A |
| 88 | 436329 | 8 | 9825154 | rs1521197 | G | A |
| 89 | 436807 | 8 | 10541651 | rs13259547 | T | G |
| 90 | 436864 | 8 | 10624854 | rs13270447 | G | T |
| 91 | 436882 | 8 | 10670108 | rs6989930 | A | G |
| 92 | 436903 | 8 | 10727072 | rs17152632 | A | G |
| 93 | 441335 | 8 | 19830921 | exm- | C | T |
| | | | | rs10096633 | | |
| 94 | 441341 | 8 | 19845376 | rs7841189 | C | T |
| 95 | 441343 | 8 | 19849757 | rs17091905 | G | A |
| 96 | 446094 | 8 | 31159672 | rs2881123 | T | C |
| 97 | 450605 | 8 | 55278457 | rs2594797 | A | G |
| 98 | 451251 | 8 | 57520600 | rs4738532 | A | G |
| 99 | 452306 | 8 | 61559253 | rs654534 | T | C |
| 100 | 452848 | 8 | 63538623 | rs16929302 | G | A |
| 101 | 457882 | 8 | 83152911 | rs1563244 | C | T |
| 102 | 459063 | 8 | 89147717 | rs10099888 | T | C |
| 103 | 460760 | 8 | 96406796 | rs3133749 | G | A |
| 104 | 465621 | 8 | 115800717 | rs16886394 | C | T |
| 105 | 471057 | 8 | 133506298 | rs2597364 | A | C |
| 106 | 471168 | 8 | 133786954 | rs1863349 | A | G |
| 107 | 471252 | 8 | 133916504 | rs6984038 | A | G |
| 108 | 472917 | 8 | 138813360 | rs6577842 | T | G |
| 109 | 484795 | 9 | 18154099 | rs1411790 | A | C |
| 110 | 486802 | 9 | 23345347 | rs11794152 | A | G |
| 111 | 487264 | 9 | 25233401 | rs10812206 | C | T |
| 112 | 490428 | 9 | 34572767 | rs10758268 | T | G |
| 113 | 503155 | 9 | 104765867 | rs1418247 | T | G |
| 114 | 520711 | 10 | 4061039 | rs10904169 | A | G |
| 115 | 520712 | 10 | 4062327 | rs7919822 | G | A |
| 116 | 531895 | 10 | 31361464 | rs7095095 | A | G |
| 117 | 531902 | 10 | 31374166 | rs12244754 | C | A |
| 118 | 534297 | 10 | 44580839 | rs268300 | G | A |
| 119 | 540501 | 10 | 67087439 | rs4746509 | A | C |
| 120 | 556304 | 10 | 109469934 | rs2251717 | C | A |
| 121 | 559356 | 10 | 118522665 | rs1665672 | G | T |
| 122 | 570866 | 11 | 5841856 | rs4758435 | C | T |
| 123 | 579054 | 11 | 23776103 | rs11820608 | A | G |
| 124 | 579056 | 11 | 23779099 | rs11827258 | C | A |
| 125 | 582325 | 11 | 35073802 | rs879488 | A | G |
| 126 | 596874 | 11 | 76054902 | rs17134811 | T | C |
| 127 | 602606 | 11 | 94667964 | exm- | G | A |
| | | | | rs10831284 | | |
| 128 | 602607 | 11 | 94667964 | rs10831284 | G | A |
| 129 | 605477 | 11 | 102675489 | rs519806 | A | G |
| 130 | 607790 | 11 | 110562578 | rs7102109 | A | G |
| 131 | 616841 | 11 | 131243929 | rs7943929 | T | G |
| 132 | 622137 | 12 | 7376438 | rs1001653 | C | T |
| 133 | 649607 | 12 | 93136257 | rs10745620 | A | G |
| 134 | 653320 | 12 | 103670917 | rs12311525 | A | G |
| 135 | 653325 | 12 | 103692799 | rs10778229 | G | T |
| 136 | 659289 | 12 | 119380724 | rs1961707 | G | A |
| 137 | 664440 | 12 | 131142464 | rs7966303 | C | T |
| 138 | 664441 | 12 | 131142912 | rs7966789 | C | T |
| 139 | 664457 | 12 | 131162850 | rs4759752 | G | A |
| 140 | 664459 | 12 | 131164658 | rs7308461 | T | C |
| 141 | 675069 | 13 | 43421683 | rs942462 | C | T |
| 142 | 676016 | 13 | 46288145 | rs7317245 | A | G |
| 143 | 676020 | 13 | 46291526 | rs9534199 | T | C |
| 144 | 677492 | 13 | 50948523 | rs2812219 | G | T |
| 145 | 678501 | 13 | 53752340 | rs1322960 | G | A |
| 146 | 678503 | 13 | 53768025 | rs2806947 | G | A |
| 147 | 680261 | 13 | 62047768 | rs7994590 | C | T |
| 148 | 680334 | 13 | 62470180 | rs17060536 | C | T |
| 149 | 680337 | 13 | 62494905 | rs1332384 | A | G |
| 150 | 680622 | 13 | 64013844 | rs616667 | A | G |
| 151 | 680687 | 13 | 64263419 | rs9570999 | G | A |
| 152 | 698795 | 14 | 22038210 | rs17792766 | G | T |
| 153 | 699804 | 14 | 23780222 | rs10149339 | A | G |
| 154 | 701849 | 14 | 28114641 | rs17112954 | T | C |
| 155 | 702427 | 14 | 30845210 | rs17096610 | G | A |
| 156 | 705032 | 14 | 39122375 | rs7141696 | T | C |
| 157 | 705371 | 14 | 40296775 | rs4313723 | C | T |
| 158 | 706724 | 14 | 46336392 | rs372543 | A | G |
| 159 | 706749 | 14 | 46474336 | rs17116830 | A | G |
| 160 | 717159 | 14 | 76573554 | rs17104036 | G | A |
| 161 | 721739 | 14 | 91199626 | rs10133270 | A | G |
| 162 | 724069 | 14 | 95879022 | rs11623037 | C | T |
| 163 | 737254 | 15 | 48178967 | rs482624 | C | A |
| 164 | 744691 | 15 | 68864495 | rs12909046 | G | A |
| 165 | 750171 | 15 | 86052670 | rs756426 | G | T |
| 166 | 765446 | 16 | 13238897 | rs150063 | T | C |
| 167 | 769859 | 16 | 26683422 | rs235807 | G | A |
| 168 | 778173 | 16 | 66457653 | rs1837024 | T | C |
| 169 | 782709 | 16 | 77959692 | rs372627 | C | T |
| 170 | 795588 | 17 | 8384077 | rs6503121 | C | T |
| 171 | 799541 | 17 | 17700573 | rs4925112 | T | C |
| 172 | 817738 | 17 | 71834172 | rs4789011 | C | T |
| 173 | 820475 | 17 | 76326637 | rs7222391 | G | A |
| 174 | 828846 | 18 | 13642721 | rs12326169 | T | C |
| 175 | 829961 | 18 | 21825411 | rs1623173 | C | A |
| 176 | 839429 | 18 | 52780909 | rs4510110 | A | G |
| 177 | 840865 | 18 | 57085544 | rs2069124 | A | C |
| 178 | 843644 | 18 | 65667017 | rs8096215 | A | G |
| 179 | 850531 | 19 | 3698842 | rs4432372 | A | G |
| 180 | 853998 | 19 | 9074950 | rs2591591 | G | T |
| 181 | 854009 | 19 | 9075969 | rs2591593 | C | T |
| 182 | 854011 | 19 | 9076083 | rs2547072 | G | A |
| 183 | 854014 | 19 | 9076278 | rs953551 | G | A |
| 184 | 854020 | 19 | 9076728 | rs2591594 | C | T |
| 185 | 854023 | 19 | 9076858 | rs2547074 | C | T |
| 186 | 854025 | 19 | 9076950 | rs2547076 | C | A |
| 187 | 854026 | 19 | 9076991 | rs2591595 | C | T |
| 188 | 854034 | 19 | 9077436 | rs1862462 | G | A |
| 189 | 854038 | 19 | 9077803 | rs2591597 | T | C |
| 190 | 854052 | 19 | 9083143 | rs7245949 | G | A |
| 191 | 854054 | 19 | 9083174 | rs7245960 | G | A |
| 192 | 854068 | 19 | 9084183 | rs1609460 | G | A |
| 193 | 854070 | 19 | 9084216 | rs1609459 | T | C |
| 194 | 854100 | 19 | 9086123 | rs10402812 | A | C |
| 195 | 875701 | 19 | 55831188 | rs2384687 | A | G |
| 196 | 882558 | 20 | 9497698 | rs8117167 | T | C |
| 197 | 882623 | 20 | 9601215 | rs219865 | T | C |
| 198 | 882625 | 20 | 9608977 | rs6039529 | A | G |
| 199 | 886487 | 20 | 19631263 | rs6081694 | G | A |
| 200 | 902925 | 21 | 15663291 | rs2822554 | C | T |
| 201 | 906128 | 21 | 25935761 | rs8134718 | C | T |
| 202 | 907670 | 21 | 30848368 | rs914136 | T | C |
| 203 | 907671 | 21 | 30848392 | rs2832341 | T | G |
| 204 | 909114 | 21 | 34652958 | rs2244305 | C | T |
| 205 | 909118 | 21 | 34660001 | rs765429 | C | T |
| 206 | 913425 | 21 | 44057708 | rs4413240 | G | A |
| 207 | 919962 | 22 | 27343892 | rs9620647 | G | A |
| 208 | 919970 | 22 | 27361417 | rs5752454 | G | A |
| 209 | 921369 | 22 | 30863392 | rs17659494 | A | C |

1. A method of treating a patient for hypothyroidism, comprising:
a) obtaining a sample from a patient containing DNA;
b) determining a number of pre-defined single nucleotide polymorphisms (SNPs) contained in the DNA of the sample;
c) administering to the patient a therapeutically effective amount of levothyroxine if the DNA of the sample contains less than a threshold number of pre-defined SNPs; or
d) administering to the patient a therapeutically effective amount of levothyroxine and a secondary therapeutic agent if the DNA of the sample contains a number of pre-defined SNPs that is equal to or greater than the threshold number of pre-defined SNPs.

2. The method of claim 1, wherein the sample is a blood sample, feces, urine, semen, saliva, hair, teeth, bone, or tissue.

3. The method of claim 1, wherein the pre-defined SNPs comprise one or more of rs2789337, rs2789334, rs1009080, exm-rs4949526, rs12059980, rs3908575, rs10157812, rs11166015, rs7519343, rs2295959, rs12468314, rs4853069, rs4508624, rs10490307, rs11896839, rs7576069, rs6757538, rs4954642, rs4664152, rs7565121, rs10204885, rs10497159, rs10191487, rs11894687, rs983733, rs6727865, exm-rs1569175, rs1569175, rs975894, rs10204710, rs3770536, rs4375854, rs6754280, rs4575672, rs962835, rs9826914, rs17023487, rs13094598, rs11720980, rs9845467, rs9859768, rs7626568, rs2197737, rs992130, rs6438269, rs7638568, rs10934299, rs9876228, rs1566695, rs7628226, rs6814444, rs3172524, rs2036204, rs13126556, rs4862815, rs12521751, rs3105807, rs2441115, rs32165, rs32153, rs6868612, rs2471106, rs1017998, rs10064975, rs1181968, rs1181962, rs13181777, rs4835970, rs2214077, rs7743224, rs1626976, rs873053, rs1948846, rs9363303, rs4537121, rs2350289, rs10498961, rs2250276, rs2787925, rs7742254, rs9489815, rs7801891, rs803157, rs1858826, rs6973845, rs855736, rs7790889, rs1521197, rs13259547, rs13270447, rs6989930, rs17152632, exm-rs10096633, rs7841189, rs17091905, rs2881123, rs2594797, rs4738532, rs654534, rs16929302, rs1563244, rs10099888, rs3133749, rs16886394, rs2597364, rs1863349, rs6984038, rs6577842, rs1411790, rs11794152, rs10812206, rs10758268, rs1418247, rs10904169, rs7919822, rs7095095, rs12244754, rs268300, rs4746509, rs2251717, rs1665672, rs4758435, rs11820608, rs11827258, rs879488, rs17134811, exm-rs10831284, rs10831284, rs519806, rs7102109, rs7943929, rs1001653, rs10745620, rs12311525, rs10778229, rs1961707, rs7966303, rs7966789, rs4759752, rs7308461, rs942462, rs7317245, rs9534199, rs2812219, rs1322960, rs2806947, rs7994590, rs17060536, rs1332384, rs616667, rs9570999, rs17792766, rs10149339, rs17112954, rs17096610, rs7141696, rs4313723, rs372543, rs17116830, rs17104036, rs10133270, rs11623037, rs482624, rs12909046, rs756426, rs150063, rs235807, rs1837024, rs372627, rs6503121, rs4925112, rs4789011, rs7222391, rs12326169, rs1623173, rs4510110, rs2069124, rs8096215, rs4432372, rs2591591, rs2591593, rs2547072, rs953551, rs2591594, rs2547074, rs2547076, rs2591595, rs1862462, rs2591597, rs7245949, rs7245960, rs1609460, rs1609459, rs10402812, rs2384687, rs8117167, rs219865, rs6039529, rs6081694, rs2822554, rs8134718, rs914136, rs2832341, rs2244305, rs765429, rs4413240, rs9620647, rs5752454, and rs17659494.

4. The method of claim 1, wherein the threshold number of pre-defined SNPs is about 15.

5. The method of claim 1, wherein the threshold number of pre-defined SNPs is about 107.

6. The method of claim 1, wherein the secondary therapeutic agent comprises one or more of liothyronine, desiccated thyroid extract, T3 sulphate or poly-zinc-liothyronine, and T3 analogues.

7. The method of claim 6, wherein the T3 analogues comprise eprotirome and sobetirome.

8. The method of claim 6, wherein the desiccated thyroid extract contains levothyroxine and liothyronine derived from an animal source.

9. The method of claim 1 further comprising:
(e) improving one or more of quality of life, mood, and cognitive parameters in the patient.

10. A method of improving treatment to a patient for hypothyroidism, comprising:
a) obtaining a sample from a patient containing DNA;
b) determining a number of pre-defined single nucleotide polymorphisms (SNPs) contained in the DNA of the sample;
c) administering to the patient a therapeutically effective amount of levothyroxine if the DNA of the sample contains less than a threshold number of pre-defined SNPs; or
d) administering to the patient a therapeutically effective amount of levothyroxine and a secondary therapeutic agent if the DNA of the sample contains a number of pre-defined SNPs that is equal to or greater than the threshold number of pre-defined SNPs; and
(e) one or more of
i) reducing incidents of the patient receiving an ineffective treatment for hypothyroidism,
ii) reducing the time required for the patient to receive an effective treatment for hypothyroidism, and
iii) reducing healthcare costs associated with receiving treatment for hypothyroidism.

11. The method of claim 10, wherein the sample is a blood sample, feces, urine, semen, saliva, hair, teeth, bone, or tissue.

12. The method of claim 10, wherein the pre-defined SNPs comprise one or more of rs2789337, rs2789334, rs1009080, exm-rs4949526, rs12059980, rs3908575, rs10157812, rs11166015, rs7519343, rs2295959, rs12468314, rs4853069, rs4508624, rs10490307, rs11896839, rs7576069, rs6757538, rs4954642, rs4664152, rs7565121, rs10204885, rs10497159, rs10191487, rs11894687, rs983733, rs6727865, exm-rs1569175, rs1569175, rs975894, rs10204710, rs3770536, rs4375854, rs6754280, rs4575672, rs962835, rs9826914, rs17023487, rs13094598, rs11720980, rs9845467, rs9859768, rs7626568, rs2197737, rs992130, rs6438269, rs7638568, rs10934299, rs9876228, rs1566695, rs7628226, rs6814444, rs3172524, rs2036204, rs13126556, rs4862815, rs12521751, rs3105807, rs2441115, rs32165, rs32153, rs6868612, rs2471106, rs1017998, rs10064975, rs1181968, rs1181962, rs13181777, rs4835970, rs2214077, rs7743224, rs1626976, rs873053, rs1948846, rs9363303, rs4537121, rs2350289, rs10498961, rs2250276, rs2787925, rs7742254, rs9489815, rs7801891, rs803157, rs1858826, rs6973845, rs855736, rs7790889, rs1521197, rs13259547, rs13270447, rs6989930, rs17152632, exm-rs10096633, rs7841189, rs17091905, rs2881123, rs2594797, rs4738532, rs654534, rs16929302, rs1563244, rs10099888, rs3133749, rs16886394, rs2597364, rs1863349, rs6984038, rs6577842, rs1411790, rs11794152, rs10812206, rs10758268, rs1418247, rs10904169, rs7919822, rs7095095, rs12244754, rs268300, rs4746509, rs2251717, rs1665672, rs4758435, rs11820608, rs11827258, rs879488, rs17134811, exm-rs10831284, rs10831284, rs519806, rs7102109, rs7943929, rs1001653, rs10745620, rs12311525, rs10778229, rs1961707, rs7966303, rs7966789, rs4759752, rs7308461, rs942462, rs7317245, rs9534199, rs2812219, rs1322960, rs2806947, rs7994590, rs17060536, rs1332384, rs616667, rs9570999, rs17792766, rs10149339, rs17112954, rs17096610, rs7141696, rs4313723, rs372543, rs17116830, rs17104036, rs10133270, rs11623037, rs482624, rs12909046, rs756426, rs150063, rs235807, rs1837024, rs372627, rs6503121, rs4925112, rs4789011, rs7222391, rs12326169, rs1623173, rs4510110, rs2069124, rs8096215, rs4432372, rs2591591, rs2591593, rs2547072, rs953551, rs2591594, rs2547074, rs2547076, rs2591595, rs1862462, rs2591597, rs7245949, rs7245960, rs1609460, rs1609459, rs10402812, rs2384687, rs8117167, rs219865, rs6039529, rs6081694, rs2822554, rs8134718, rs914136, rs2832341, rs2244305, rs765429, rs4413240, rs9620647, rs5752454, and rs17659494.

13. The method of claim 10, wherein the threshold number of pre-defined SNPs is about 15.

14. The method of claim 10, wherein the threshold number of pre-defined SNPs is about 107.

15. The method of claim 10, wherein the secondary therapeutic agent comprises one or more of liothyronine, desiccated thyroid extract, T3 sulphate or poly-zinc-liothyronine, and T3 analogues.

16. The method of claim 15, wherein the T3 analogues comprise eprotirome and sobetirome.

17. The method of claim 15, wherein the desiccated thyroid extract contains levothyroxine and liothyronine derived from an animal source.

18. A method of treating a patient for hypothyroidism, comprising:
a) obtaining a sample from a patient containing DNA;
b) determining a number of pre-defined single nucleotide polymorphisms (SNPs) contained in the DNA of the sample;
c) administering to the patient a therapeutically effective amount of levothyroxine if the DNA sample contains less than a threshold number of pre-defined SNPs; or
d) administering to the patient a therapeutically effective amount of levothyroxine and a secondary therapeutic agent if the DNA of the sample contains a number of pre-defined SNPs that is equal to or greater than the threshold number of pre-defined SNPs,
wherein when the number of pre-defined SNPs in the sample is greater than the threshold number of pre-defined SNPs, the number of pre-defined SNPs present in the DNA of the sample correlates to a relative dose of the secondary therapeutic agent required to effectively treat hypothyroidism in the patient.
